(19) United States
(12) Patent Application Publication
(10) Pub. No.: US 2008/0227783 A1
(43) Pub. Date: Sep. 18, 2008

(54) 5-Substituted Thiazol-2-Yl Amino Compounds and Compositions as Protein Kinase Inhibitors

(75) Inventors: Yongqin Wan, Irvine, CA (US); Nathanael S. Gray, Boston, MA (US)

Correspondence Address: GENOMICS INSTITUTE OF THE;NOVARTIS RESEARCH FOUNDATION, 10675 JOHN JAY HOPKINS DRIVE, SUITE E225, SAN DIEGO, CA, 92121-1127, US

(73) Assignee: IRM LLC, Hamilton, BM (BM)

(21) Appl. No.: 11/996,743

(22) PCT Filed: Jul. 25, 2006
(86) PCT No.: PCT/US2006/029162

§ 371 Date: Jan. 24, 2008

Related U.S. Application Data
(60) Provisional application No. 60/704,976, filed on Aug. 2, 2005.

(51) Int. Cl. A61K 31/426; C07D 277/20; A61K 31/5377; A61P 25/00; C07D 413/12

(52) U.S. Cl. 514/236.8; 548/190; 548/198; 544/133; 514/370

(57) ABSTRACT

The invention provides 5-substituted thiazol-2-yl amino compounds of Formula (I): pharmaceutical compositions comprising such compounds and the use of such compounds for the treatment or prevention of diseases or disorders associated with abnormal or deregulated kinase activity, particularly diseases or disorders that involve abnormal activation of Ab 1, Aurora-A, Ber-Ab1, Bmx, CDKI/cyclinB, CHK2, Fes, FGFR3, Flt3, GSK3β, JNK1α1, Lck, MKK4 and TrkB kinases.

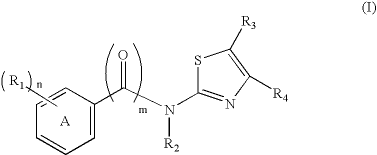

The novel compounds of this invention inhibit the activity of one or more protein kinases and are, therefore, expected to be useful in the treatment of kinase-associated diseases.

### SUMMARY OF THE INVENTION

In one aspect, the present invention provides compounds of Formula I:

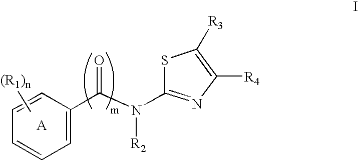

in which:

n is selected from 0, 1, 2 and 3;

m is selected from 0 and 1;

R₁ is selected from halo, cyano, hydroxy, nitro, C₁₋₆alkyl, C₁₋₆alkoxy, halo-substituted-C₁₋₆alkyl, halo-substituted-C₁₋₆alkoxy, —S(O)₀₋₂R₅, —NR₅R₅, —C(O)NR₅R₆, —C(O)NR₅R₆, —C(O)NR₅XOR₅, —C(O)NR₅XNR₅R₅, —OR₆, —C(O)OR₅, —NR₅C(O)R₆; wherein each R₅ is independently selected from hydrogen and C₁₋₆alkyl; and R₆ is selected from C₆₋₁₀aryl-C₀₋₄alkyl, C₁₋₁₀heteroaryl-C₀₋₄alkyl, C₃₋₁₂cycloalkyl-C₀₋₄alkyl and C₃₋₈heterocycloalkyl-C₀₋₄alkyl; or where n is 2, two adjacent R₁ radicals together with the atoms to which they are both attached, form phenyl, such that ring A becomes optionally substituted naphthyl (for example compound 59 of table 1, infra);

R₂ is hydrogen and methyl;

R₃ is halo;

R₄ is selected from hydrogen, halogen and C₁₋₆alkyl; or R₃ and R₄ together with the atoms to which R₃ and R₄ are attached form phenyl; and phenyl ring A can optionally have up to three ═C— groups replaced with ═N—; and the N-oxide derivatives, prodrug derivatives, protected derivatives, individual isomers and mixture of isomers thereof; and the pharmaceutically acceptable salts and solvates (e.g. hydrates) of such compounds.

In a second aspect, the present invention provides a pharmaceutical composition which contains a compound of Formula I or a N-oxide derivative, individual isomers and mixture of isomers thereof; or a pharmaceutically acceptable salt thereof, in admixture with one or more suitable excipients.

In a third aspect, the present invention provides a method of treating a disease in an animal in which inhibition of kinase activity, particularly Abl, Aurora-A, Bcr-Abl, Bmx, CDK1/cyclinB, CHK2, Fes, FGFR3, Flt3, GSK3β, JNK1α1, Lck, MKK4 and/or TrkB activity, can prevent, inhibit or ameliorate the pathology and/or symptomology of the diseases, which method comprises administering to the animal a therapeutically effective amount of a compound of Formula I or a N-oxide derivative, individual isomers and mixture of isomers thereof, or a pharmaceutically acceptable salt thereof.

In a fourth aspect, the present invention provides the use of a compound of Formula I in the manufacture of a medicament for treating a disease in an animal in which kinase activity, particularly Abl, Aurora-A, Bcr-Abl, Bmx, CDK1/cyclinB, CHK2, Fes, FGFR3, Flt3, GSK3β, JNK1α1, Lck, MKK4 and/or TrkB activity, contributes to the pathology and/or symptomology of the disease.

In a fifth aspect, the present invention provides a process for preparing compounds of Formula I and the N-oxide derivatives, prodrug derivatives, protected derivatives, individual isomers and mixture of isomers thereof, and the pharmaceutically acceptable salts thereof.

### DETAILED DESCRIPTION OF THE INVENTION

### Definitions

“Alkyl” as a group and as a structural element of other groups, for example halo-substituted-alkyl and alkoxy, can be either straight-chained or branched. C₁₋₄-alkoxy includes, methoxy, ethoxy, and the like. Halo-substituted alkyl includes trifluoromethyl, pentafluoroethyl, and the like.

“Aryl” means a monocyclic or fused bicyclic aromatic ring assembly containing six to ten ring carbon atoms. For example, aryl may be phenyl or naphthyl, preferably phenyl. “Arylene” means a divalent radical derived from an aryl group.

“Heteroaryl” is as defined for aryl above where one or more of the ring members is a heteroatom. For example C₁₋₁₀heteroaryl, as used in this application, includes pyridyl, indolyl, indazolyl, quinoxalinyl, quinolinyl, benzofuranyl, benzopyranyl, benzothiopyranyl, benzo[1,3]dioxole, imidazolyl, benzo-imidazolyl, pyrimidinyl, furanyl, oxazolyl, isoxazolyl, triazolyl, tetrazolyl, pyrazolyl, thienyl, etc.

“Cycloalkyl” means a saturated or partially unsaturated, monocyclic, fused bicyclic or bridged polycyclic ring assembly containing the number of ring atoms indicated. For example, C₃₋₁₀cycloalkyl includes cyclopropyl, cyclobutyl, cyclopentyl, cyclohexyl, etc.

“Heterocycloalkyl” means cycloalkyl, as defined in this application, provided that one or more of the ring carbons indicated, are replaced by a moiety selected from —O—, —N═, —NR—, —C(O)—, —S—, —S(O)— or —S(O)₂—, wherein R is hydrogen, C₁₋₄alkyl or a nitrogen protecting group. For example, C₃₋₈heterocycloalkyl as used in this application to describe compounds of the invention includes morpholino, pyrrolidinyl, pyrrolidinyl-2-one, piperazinyl, piperidinyl, piperidinylone, 1,4-dioxa-8-aza-spiro[4.5]dec-8-yl, etc.

“Halogen” (or halo) preferably represents chloro or fluoro, but may also be bromo or iodo.

“Kinase Panel” is a list of kinases comprising Abl(human), Abl(T315I), JAK2, JAK3, ALK, JNK1α1, ALK4, KDR, Aurora-A, Lck, Blk, MAPK1, Bmx, MAPKAP-K2, BRK, MEK1, CaMKII(rat), Met, CDK1/cyclinB, p70S6K, CHK2, PAK2, CK1, PDGFRα, CK2, PDK1, c-kit, Pim-2, c-RAF, PKA(h), CSK, PKBα, cSrc, PKCα, DYRK2, Plk3, EGFR, ROCK-I, Fes, Ron, FGFR3, Ros, Flt3, SAPK2α, Fms, SGK, Fyn, SIK, GSK3β, Syk, IGF-1R, Tie-2, IKKβ, TrKB, IR, WNK3, IRAK4, ZAP-70, ITK, AMPK(rat), LIMK1, Rsk2, Axl, LKB1, SAPK2β, BrSK2, Lyn (h), SAPK3, BTK, MAPKAP-K3, SAPK4, CaMKIV, MARK1, Snk, CDK2/cyclinA, MINK, SRPK1, CDK3/cyclinE, MKK4(m), TAK1, CDK5/p25, MKK6(h), TBK1, CDK6/cyclinD3, MLCK, TrkA, CDK7/cyclinH/MAT1, MRCKβ, TSSK1, CHK1, MSK1, Yes, CK1d, MST2, ZIPK, c-Kit (D816V), MuSK, DAPK2, NEK2, DDR2, NEK6, DMPK, PAK4, DRAK1, PAR-1Bα, EphA1, PDGFRβ, EphA2, Pim-1, EphA5, PKBβ, EphB2, PKCβI, EphB4, PKCδ, FGFR1, PKCη, FGFR2, PKCθ, FGFR4, PKD2, Fgr, PKG1β, Flt1, PRK2, Hck, PYK2, HIPK2, Ret, IKKα, RIPK2, IRR, ROCK-II(human), JNK2α2, Rse, JNK3, Rsk1(h), PI3 Kγ, PI3 Kδ and PI3-Kβ. Compounds of the invention are screened against the kinase panel (wild type and/or mutation thereof) and inhibit the activity of at least one of said panel members.

“Mutant forms of BCR-Abl” means single or multiple amino acid changes from the wild-type sequence. Mutations in BCR-ABL act by disrupting critical contact points between protein and inhibitor (for example, Gleevec, and the like), more often, by inducing a transition from the inactive to the active state, i.e. to a conformation to which BCR-ABL and Gleevec is unable to bind. From analyses of clinical samples, the repertoire of mutations found in association with the resistant phenotype has been increasing slowly but inexorably over time. Mutations seem to cluster in four main regions. One group of mutations (G250E, Q252R, Y253F/H, E255K/V) includes amino acids that form the phosphate-binding loop for ATP (also known as the P-loop). A second group (V289A, F311L, T315I, F317L) can be found in the Gleevec binding site and interacts directly with the inhibitor via hydrogen bonds or Van der Waals' interactions. The third group of mutations (M351T, E355G) clusters in close proximity to the catalytic domain. The fourth group of mutations (H396R/P) is located in the activation loop, whose conformation is the molecular switch controlling kinase activation/inactivation. BCR-ABL point mutations associated with Gleevec resistance detected in CML and ALL patients include: M224V, L248V, G250E, G250R, Q252R, Q252H, Y253H, Y253F, E255K, E255V, D276G, T277A, V289A, F311L, T315I, T315N, F317L, M343T, M315T, E355G, F359V, F359A, V379I, F382L, L387M, L387F, H396P, H396R, A397P, S417Y, E459K, and F486S (Amino acid positions, indicated by the single letter code, are those for the GenBank sequence, accession number AAB60394, and correspond to ABL type 1a; Martinelli et al., Haematologica/The Hematology Journal, 2005, April; 90-4). Unless otherwise stated for this invention, Bcr-Abl refers to wild-type and mutant forms of the enzyme.

“Treat”, “treating” and “treatment” refer to a method of alleviating or abating a disease and/or its attendant symptoms.

### Description of the Preferred Embodiments

The fusion protein BCR-Abl is a result of a reciprocal translocation that fuses the Abl proto-oncogene with the Bcr gene. BCR-Abl is then capable of transforming B-cells through the increase of mitogenic activity. This increase results in a reduction of sensitivity to apoptosis, as well as altering the adhesion and homing of CML progenitor cells. The present invention provides compounds, compositions and methods for the treatment of kinase related disease, particularly Abl, Aurora-A, Bcr-Abl, Bmx, CDK1/cyclinB, CHK2, Fes, FGFR3, Flt3, GSK3β, JNK1α1, Lck, MKK4 and TrkB kinase related diseases. For example, leukemia and other proliferation disorders related to BCR-Abl can be treated through the inhibition of wild type and mutant forms of Bcr-Abl.

In one embodiment, with reference to compounds of Formula I, ring A is selected from phenyl, pyridinyl and naphthyl (where 2 radicals of R₁ combine to form a phenyl ring fused to ring A, thereby creating naphthyl); m is zero; R₃ is halo and R₄ is hydrogen.

In another embodiment, R₁ is selected from methyl, hydroxy, methoxy, chloro, fluoro, bromo, carboxy, amino, cyano, nitro, methyl-sulfanyl, trifluoromethoxy, trifluoromethyl, methyl-carbonyl, ethoxy-carbonyl, —C(O)NHR₆, —C(O)NH(CH₂)₂OCH₃, —C(O)NHCH(CH₃)CH₂OCH₃, —C(O)N(CH₃)(CH₂)₂OCH₃, —C(O)NH(CH₂)₂OH, —C(O)NH(CH₂)₂N(CH₃)₂, —C(O)NH(CH₂)₂N(C₂H₅)₂, —C(O)NHCH₃, —NHC(O)R₆, —NHC(O)CH₃ and —OR₆; wherein R₆ is selected from phenyl, morpholino-ethyl, pyridinyl and pyrrolidinyl-ethyl.

Preferred compounds of the invention are selected from: (5-Bromo-thiazol-2-yl)-p-tolyl-amine; 4-(5-Bromo-thiazol-2-ylamino)-phenol; (5-Bromo-thiazol-2-yl)-(4-methoxy-phenyl)-amine; 4-(5-Bromo-thiazol-2-ylamino)-benzoic acid; 4-(5-Bromo-thiazol-2-ylamino)-N-(2-morpholin-4-yl-ethyl)-benzamide; 4-(5-Bromo-thiazol-2-ylamino)-N-(2-methoxy-ethyl)-benzamide; (5-Bromo-thiazol-2-yl)-[4-(1-methylamino-vinyl)-phenyl]-amine; 3-(5-Bromo-thiazol-2-ylamino)-benzoic acid; N-[4-(5-Bromo-thiazol-2-ylamino)-phenyl]-benzamide; N-[4-(5-Bromo-thiazol-2-ylamino)-phenyl]-acetamide; 3-(5-Bromo-thiazol-2-ylamino)-N-(2-methoxy-ethyl)-benzamide; 3-(5-Bromo-thiazol-2-ylamino)-N-methyl-benzamide; (5-Bromo-thiazol-2-yl)-[4-(pyridin-4-yloxy)-phenyl]-amine; (5-Bromo-thiazol-2-yl)-(4-chloro-phenyl)-amine; Benzothiazol-2-yl-(4-fluoro-phenyl)-amine; 4-(5-Bromo-thiazol-2-ylamino)-N-(2-hydroxy-ethyl)-benzamide; 4-(5-Bromo-thiazol-2-ylamino)-N-(2-dimethylamino-ethyl)-benzamide; 4-(5-Bromo-thiazol-2-ylamino)-N-(2-diethylamino-ethyl)-benzamide; N-(5-Bromo-thiazol-2-yl)-benzamide; (5-Bromo-thiazol-2-yl)-(3-fluorophenyl)-amine; (5-Bromo-thiazol-2-yl)-(3-trifluoromethyl-phenyl)-amine; (5-Bromo-thiazol-2-yl)-(3-methoxy-phenyl)-amine; (5-Bromo-thiazol-2-yl)-m-tolyl-amine; (5-Bromo-thiazol-2-yl)-pyridin-2-yl-amine; N-(5-Bromo-thiazol-2-yl)-benzene-1,4-diamine; 1-[4-(5-Bromo-thiazol-2-ylamino)-phenyl]-ethanone; 4-(5-Bromo-thiazol-2-ylamino)-benzoic acid ethyl ester; (5-Bromo-thiazol-2-yl)-pyridin-4-yl-amine; (5-Bromo-thiazol-2-yl)-pyridin-3-yl-amine; N-(5-Bromo-thiazol-2-yl)-benzamide; (5-Bromo-thiazol-2-yl)-(4-trifluoromethyl-phenyl)-amine; 3-(5-Bromo-thiazol-2-ylamino)-benzoic acid ethyl ester; (5-Bromo-thiazol-2-yl)-phenyl-amine; (5-Bromo-thiazol-2-yl)-(2-methoxy-phenyl)-amine; (5-Bromo-thiazol-2-yl)-(4-fluoro-phenyl)-amine; (5-Chloro-thiazol-2-yl)-(4-fluoro-phenyl)-amine; (4-Fluoro-phenyl)-(5-iodo-thiazol-2-yl)-amine; 4-(5-Bromo-thiazol-2-ylamino)-benzonitrile; (5-Bromo-thiazol-2-yl)-o-tolyl-amine; (5-Bromo-thiazol-2-yl)-naphthalen-1-yl-amine; (5-Bromo-thiazol-2-yl)-(2-fluoro-phenyl)-amine; 3-(5-Bromo-thiazol-2-ylamino)-benzonitrile; (5-Bromo-thiazol-2-yl)-(3-methylsulfanyl-phenyl)-amine; (4-Bromo-phenyl)-(5-bromo-thiazol-2-yl)-amine; (5-Bromo-thiazol-2-yl)-(4-phenoxy-phenyl)-amine; (5-Bromo-thiazol-2-yl)-(4-nitro-phenyl)-amine; 4-(5-Bromo-thiazol-2-ylamino)-N-(2-pyrrolidin-1-yl-ethyl)-benzamide; 4-(5-Bromo-thiazol-2-ylamino)-N-(2-methoxy-1-methyl-ethyl)-benzamide; and 4-(5-Bromo-thiazol-2-ylamino)-N-(2-methoxy-ethyl)-N-methyl-benzamide.

Further preferred compounds of the invention are detailed in the Examples and Table I, infra.

### Pharmacology and Utility

Compounds of the invention modulate the activity of kinases and, as such, are useful for treating diseases or disorders in which kinases, contribute to the pathology and/or symptomology of the disease. Examples of kinases that are inhibited by the compounds and compositions described herein and against which the methods described herein are useful include, but are not limited to, Abl, Aurora-A, Bcr-Abl (wild-type and mutant forms), Bmx, CDK1/cyclinB, CHK2, Fes, FGFR3, Flt3, GSK3β, JNK1α1, Lck, MKK4 and TrkB

Abelson tyrosine kinase (i.e. Abl, c-Abl) is involved in the regulation of the cell cycle, in the cellular response to genotoxic stress, and in the transmission of information about the cellular environment through integrin signaling. Overall, it appears that the Abl protein serves a complex role as a cellular module that integrates signals from various extracellular and intracellular sources and that influences decisions in regard to cell cycle and apoptosis. Abelson tyrosine kinase includes sub-types derivatives such as the chimeric fusion (oncoprotein) BCR-Abl with deregulated tyrosine kinase activity or the v-Abl. BCR-Abl is critical in the pathogenesis of 95% of chronic myelogenous leukemia (CML) and 10% of acute lymphocytic leukemia. STI-571 (Gleevec) is an inhibitor of the oncogenic BCR-Abl tyrosine kinase and is used for the treatment of chronic myeloid leukemia (CML). However, some patients in the blast crisis stage of CML are resistant to STI-571 due to mutations in the BCR-Abl kinase. Over 22 mutations have been reported to date with the most common being G250E, E255V, T315I, F317L and M351T.

Compounds of the present invention inhibit abl kinase, especially v-abl kinase. The compounds of the present invention also inhibit wild-type BCR-Abl kinase and mutations of BCR-Abl kinase and are thus suitable for the treatment of Bcr-abl-positive cancer and tumor diseases, such as leukemias (especially chronic myeloid leukemia and acute lymphoblastic leukemia, where especially apoptotic mechanisms of action are found), and also shows effects on the subgroup of leukemic stem cells as well as potential for the purification of these cells in vitro after removal of said cells (for example, bone marrow removal) and reimplantation of the cells once they have been cleared of cancer cells (for example, reimplantation of purified bone marrow cells).

Certain families of protein kinases are involved in regulating centrosome and spindle function such as the Nima related kinase (Nek) 2, Polo-like kinase (Plk) 1, and Aurora kinases. Aurora kinases were first discovered in a yeast screen for mutants that displayed improper ploidy (ipl) following cell division. In Drosophila, mutations in Aurora kinase were found to prevent centrosome separation thereby resulting in monopolar spindles. There are three known isoforms of Aurora kinase described in mammals, Aurora A, B and C. While Aurora A and B are ubiquitously expressed, Aurora C shows predominant expressed in the testis suggesting a possible role is meiosis. Aurora A is localized to the centrosome and spindle poles from late S and early G2 through M phase. Aurora A binds to and is activated by TPX2 at the G2/M transition which targets Aurora A to the mitotic spindles. Aurora A can phosphorylate histone H3 on serine 10 during centrosome maturation and spindle assembly. Aurora B is a chromosome passenger protein that moves from centromeres to the spindle midzone during mitosis. Aurora B is located at the central spindles during late anaphase and at the midbody during telophase and cytokinesis. Aurora B is proposed to regulate chromosome condensation and cohesion, bipolar chromosome attachment, the spindle checkpoint and chromosome segregation. Although less is known about the importance of Aurora C, it can complement some of the functions of Aurora B.

Aurora A is located at chromosome 20q13 in a region commonly found to be genetically amplified in breast and colon cancer (protein overexpression is also detected) and is associated with a poor prognosis. Both Aurora A and B have the ability to transform cell lines (NIH3T3 or CHO) which are then capable of forming tumors in mice. The role of the Aurora kinases in cell cycle and tumorigenesis has made them potential targets for the development of small molecule therapeutics. For example, Aurora-A activity is elevated in bladder, breast, cervical, colorectal, gastric, neuroblastoma, ovarian and pancreatic cancers.

The trk family of neurotrophin receptors (trkA, trkB, trkc) promotes the survival, growth and differentiation of the neuronal and non-neuronal tissues. The TrkB protein is expressed in neuroendocrine-type cells in the small intestine and colon, in the alpha cells of the pancreas, in the monocytes and macrophages of the lymph nodes and of the spleen, and in the granular layers of the epidermis (Shibayama and Koizumi, 1996). Expression of the TrkB protein has been associated with an unfavorable progression of Wilms tumors and of neuroblastomas. TkrB is, moreover, expressed in cancerous prostate cells but not in normal cells. The signaling pathway downstream of the trk receptors involves the cascade of MAPK activation through the Shc, activated Ras, ERK-1 and ERK-2 genes, and the PLC-gamma1 transduction pathway (Sugimoto et al., 2001).

The Tec family kinase, Bmx, a non-receptor protein-tyrosine kinase, controls the proliferation of mammary epithelial cancer cells.

Fibroblast growth factor receptor 3 was shown to exert a negative regulatory effect on bone growth and an inhibition of chondrocyte proliferation. Thanatophoric dysplasia is caused by different mutations in fibroblast growth factor receptor 3, and one mutation, TDII FGFR3, has a constitutive tyrosine kinase activity which activates the transcription factor Stat1, leading to expression of a cell-cycle inhibitor, growth arrest and abnormal bone development (Su et al., Nature, 1997, 386, 288-292). FGFR3 is also often expressed in multiple myeloma-type cancers. Inhibitors of FGFR3 activity are useful in the treatment of T-cell mediated inflammatory or autoimmune diseases including but not limited to rheumatoid arthritis (RA), collagen II arthritis, multiple sclerosis (MS), systemic lupus erythematosus (SLE), psoriasis, juvenile onset diabetes, Sjogren's disease, thyroid disease, sarcoidosis, autoimmune uveitis, inflammatory bowel disease (Crohn's and ulcerative colitis), celiac disease and myasthenia gravis.

Lck plays a role in T-cell signaling. Mice that lack the Lck gene have a poor ability to develop thymocytes. The function of Lck as a positive activator of T-cell signaling suggests that Lck inhibitors may be useful for treating autoimmune disease such as rheumatoid arthritis.

JNKs, along with other MAPKs, have been implicated in having a role in mediating cellular response to cancer, thrombin-induced platelet aggregation, immunodeficiency disorders, autoimmune diseases, cell death, allergies, osteoporosis and heart disease. The therapeutic targets related to activation of the JNK pathway include chronic myelogenous leukemia (CML), rheumatoid arthritis, asthma, osteoarthritis, ischemia, cancer and neurodegenerative diseases. As a result of the importance of JNK activation associated with liver disease or episodes of hepatic ischemia, compounds of the invention may also be useful to treat various hepatic disorders. A role for JNK in cardiovascular disease such as myocardial infarction or congestive heart failure has also been reported as it has been shown JNK mediates hypertrophic responses to various forms of cardiac stress. It has been demonstrated that the JNK cascade also plays a role in T-cell activation, including activation of the IL-2 promoter. Thus, inhibitors of JNK may have therapeutic value in altering pathologic immune responses. A role for JNK activation in various cancers has also been established, suggesting the potential use of JNK inhibitors in cancer. For example, constitutively activated JNK is associated with HTLV-1 mediated tumorigenesis [Oncogene 13:135-42 (1996)]. JNKmayplayaroleinKaposi's sarcoma (KS). Other proliferative effects of other cytokines implicated in KS proliferation, such as vascular endothelial growth factor (VEGF), IL-6 and TNFα, may also be mediated by JNK. In addition, regulation of the c-jun gene in p210 BCR-ABL transformed cells corresponds with activity of JNK, suggesting a role for JNK inhibitors in the treatment for chronic myelogenous leukemia (CML) [Blood 92:2450-60 (1998)].

Mitogen-activated protein kinases (MAPKs) are members of conserved signal transduction pathways that activate transcription factors, translation factors and other target molecules in response to a variety of extracellular signals. MAPKs are activated by phosphorylation at a dual phosphorylation motif having the sequence Thr-X-Tyr by mitogen-activated protein kinase kinases (MKKs). In higher eukaryotes, the physiological role of MAPK signaling has been correlated with cellular events such as proliferation, oncogenesis, development and differentiation. Accordingly, the ability to regulate signal transduction via these pathways (particularly via MKK4 and MKK6) could lead to the development of treatments and preventive therapies for human diseases associated with MAPK signaling, such as inflammatory diseases, autoimmune diseases and cancer.

The SAPK's (also called “jun N-terminal kinases” or “JNK's”) are a family of protein kinases that represent the penultimate step in signal transduction pathways that result in activation of the c-jun transcription factor and expression of genes regulated by c-jun. In particular, c-jun is involved in the transcription of genes that encode proteins involved in the repair of DNA that is damaged due to genotoxic insults. Agents that inhibit SAPK activity in a cell prevent DNA repair and sensitize the cell to those cancer therapeutic modalities that act by inducing DNA damage.

CHK2 is a member of the checkpoint kinase family of serine/threonine protein kinases and is involved in a mechanism used for surveillance of DNA damage, such as damage caused by environmental mutagens and endogenous reactive oxygen species. As a result, it is implicated as a tumor suppressor and target for cancer therapy.

Fes is a non-receptor protein tyrosine kinase that has been implicated in a variety of cytokine signal transduction pathways, as well as differentiation of myeloid cells. Fes is also a key component of the granulocyte differentiation machinery.

Flt3 receptor tyrosine kinase activity is implicated in leukemias and myelodysplastic syndrome. In approximately 25% of AML the leukemia cells express a constitutively active form of auto-phosphorylated (p) FLT3 tyrosine kinase on the cell surface. The activity of p-FLT3 confers growth and survival advantage on the leukemic cells. Patients with acute leukemia, whose leukemia cells express p-FLT3 kinase activity, have a poor overall clinical outcome. Inhibition of p-FLT3 kinase activity induces apoptosis (programmed cell death) of the leukemic cells.

In accordance with the foregoing, the present invention further provides a method for preventing or treating any of the diseases or disorders described above in a subject in need of such treatment, which method comprises administering to said subject a therapeutically effective amount (See, “Administration and Pharmaceutical Compositions”, infra) of a compound of Formula I or a pharmaceutically acceptable salt thereof. For any of the above uses, the required dosage will vary depending on the mode of administration, the particular condition to be treated and the effect desired.

### Administration and Pharmaceutical Compositions

In general, compounds of the invention will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g. humans, is in the range from about 0.5 mg to about 100 mg, conveniently administered, e.g. in divided doses up to four times a day or in retard form. Suitable unit dosage forms for oral administration comprise from ca. 1 to 50 mg active ingredient.

Compounds of the invention can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a compound of the present invention in free form or in a pharmaceutically acceptable salt form in association with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound of the present invention with a carrier. A carrier can include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Compounds of the invention can be administered in therapeutically effective amounts in combination with one or more therapeutic agents (pharmaceutical combinations). For example, synergistic effects can occur with other immunomodulatory or anti-inflammatory substances, for example when used in combination with cyclosporin, rapamycin, or ascomycin, or immunosuppressant analogues thereof, for example cyclosporin A (CsA), cyclosporin G, FK-506, rapamycin, or comparable compounds, corticosteroids, cyclophosphamide, azathioprine, methotrexate, brequinar, leflunomide, mizoribine, mycophenolic acid, mycophenolate mofetil, 15-deoxyspergualin, immunosuppressant antibodies, especially monoclonal antibodies for leukocyte receptors, for example MHC, CD2, CD3, CD4, CD7, CD25, CD28, B7, CD45, CD58 or their ligands, or other immunomodulatory compounds, such as CTLA41g. Where the compounds of the invention are administered in conjunction with other therapies, dosages of the co-administered compounds will of course vary depending on the type of co-drug employed, on the specific drug employed, on the condition being treated and so forth.

The invention also provides for a pharmaceutical combinations, e.g. a kit, comprising a) a first agent which is a compound of the invention as disclosed herein, in free form or in pharmaceutically acceptable salt form, and b) at least one co-agent. The kit can comprise instructions for its administration.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g. a compound of Formula I and a co-agent, are both administered to a patient simultaneously in the form of a single entity or dosage. The term “non-fixed combination” means that the active ingredients, e.g. a compound of Formula I and a co-agent, are both administered to a patient as separate entities either simultaneously, concurrently or sequentially with no specific time limits, wherein such administration provides therapeutically effective levels of the 2 compounds in the body of the patient. The latter also applies to cocktail therapy, e.g. the administration of 3 or more active ingredients.

### Processes for Making Compounds of the Invention

The present invention also includes processes for the preparation of compounds of the invention. In the reactions described, it can be necessary to protect reactive functional groups, for example hydroxy, amino, imino, thio or carboxy groups, where these are desired in the final product, to avoid their unwanted participation in the reactions. Conventional protecting groups can be used in accordance with standard practice, for example, see T. W. Greene and P. G. M. Wuts in “Protective Groups in Organic Chemistry”, John Wiley and Sons, 1991.

Compounds of Formula I can be prepared by proceeding as in the following Reaction Scheme I:

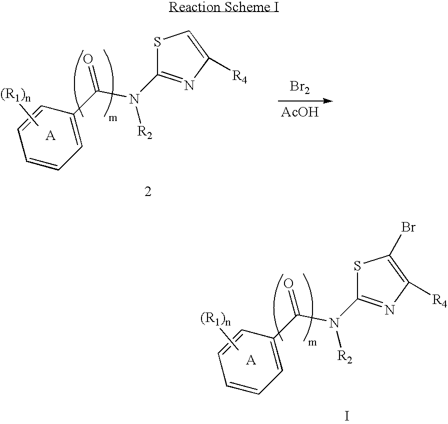

in which n, m, R₁, R₂ and R₄ are as defined in the Summary of the Invention. R₃, in this instance is Br, but can be Cl or I depending on the reactants used. A compound of Formula I can be synthesized by reacting a compound of formula 2 in the presence of a suitable solvent (for example, AcOH, and the like), and bromine. The reaction proceeds in a temperature range of about 0° C. to about 40° C. and can take up to about 10 hours to complete.

Detailed examples of the synthesis of a compound of Formula I can be found in the Examples, infra.

### Additional Processes for Making Compounds of the Invention

A compound of the invention can be prepared as a pharmaceutically acceptable acid addition salt by reacting the free base form of the compound with a pharmaceutically acceptable inorganic or organic acid. Alternatively, a pharmaceutically acceptable base addition salt of a compound of the invention can be prepared by reacting the free acid form of the compound with a pharmaceutically acceptable inorganic or organic base.

Alternatively, the salt forms of the compounds of the invention can be prepared using salts of the starting materials or intermediates.

The free acid or free base forms of the compounds of the invention can be prepared from the corresponding base addition salt or acid addition salt from, respectively. For example a compound of the invention in an acid addition salt form can be converted to the corresponding free base by treating with a suitable base (e.g., ammonium hydroxide solution, sodium hydroxide, and the like). A compound of the invention in a base addition salt form can be converted to the corresponding free acid by treating with a suitable acid (e.g., hydrochloric acid, etc.).

Compounds of the invention in unoxidized form can be prepared from N-oxides of compounds of the invention by treating with a reducing agent (e.g., sulfur, sulfur dioxide, triphenyl phosphine, lithium borohydride, sodium borohydride, phosphorus trichloride, tribromide, or the like) in a suitable inert organic solvent (e.g. acetonitrile, ethanol, aqueous dioxane, or the like) at 0 to 80° C.

Prodrug derivatives of the compounds of the invention can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the invention with a suitable carbamylating agent (e.g., 1,1-acyloxyalkylcarbanochloridate, para-nitrophenyl carbonate, or the like).

Protected derivatives of the compounds of the invention can be made by means known to those of ordinary skill in the art. A detailed description of techniques applicable to the creation of protecting groups and their removal can be found in T. W. Greene, “Protecting Groups in Organic Chemistry”, 3^{rd} edition, John Wiley and Sons, Inc., 1999.

Compounds of the present invention can be conveniently prepared, or formed during the process of the invention, as solvates (e.g., hydrates). Hydrates of compounds of the present invention can be conveniently prepared by recrystallization from an aqueous/organic solvent mixture, using organic solvents such as dioxin, tetrahydrofuran or methanol.

Compounds of the invention can be prepared as their individual stereoisomers by reacting a racemic mixture of the compound with an optically active resolving agent to form a pair of diastereoisomeric compounds, separating the diastereomers and recovering the optically pure enantiomers. While resolution of enantiomers can be carried out using covalent diastereomeric derivatives of the compounds of the invention, dissociable complexes are preferred (e.g., crystalline diastereomeric salts). Diastereomers have distinct physical properties (e.g., melting points, boiling points, solubilities, reactivity, etc.) and can be readily separated by taking advantage of these dissimilarities. The diastereomers can be separated by chromatography, or preferably, by separation/resolution techniques based upon differences in solubility. The optically pure enantiomer is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions”, John Wiley And Sons, Inc., 1981.

In summary, the compounds of Formula I can be made by a process, which involves:

(a) that of reaction scheme I; and

(b) optionally converting a compound of the invention into a pharmaceutically acceptable salt;

(c) optionally converting a salt form of a compound of the invention to a non-salt form;

(d) optionally converting an unoxidized form of a compound of the invention into a pharmaceutically acceptable N-oxide;

(e) optionally converting an N-oxide form of a compound of the invention to its unoxidized form;

(f) optionally resolving an individual isomer of a compound of the invention from a mixture of isomers;

(g) optionally converting a non-derivatized compound of the invention into a pharmaceutically acceptable prodrug derivative; and

(h) optionally converting a prodrug derivative of a compound of the invention to its non-derivatized form.

Insofar as the production of the starting materials is not particularly described, the compounds are known or can be prepared analogously to methods known in the art or as disclosed in the Examples hereinafter.

One of skill in the art will appreciate that the above transformations are only representative of methods for preparation of the compounds of the present invention, and that other well known methods can similarly be used.

### EXAMPLES

The present invention is further exemplified, but not limited, by the following examples that illustrate the preparation of compounds of Formula I according to the invention.

Unless otherwise noted, materials are obtained from commercial suppliers and are used without purification. Removal of solvent under reduced pressure refers to distillation using a Büchi rotary evaporator attached to a vacuum pump (˜3 mmHg). Products obtained as solids or high boiling oils are dried under vacuum (˜1 mmHg).

Reversed-phase high-pressure liquid chromatography (HPLC) is performed using Varian Chromatography System (ProStar Model 210) with C₁₈ column (Phenomenex) using water-acetonitrile (0.035% TFA) as an eluent. ¹H NMR spectra are recorded on Bruker XWIN-NMR (400 MHz or 600 MHz). Proton resonances are reported in parts per million (ppm) down field from tetramethylsilane (TMS). ¹H NMR data are reported as multiplicity (s singlet, d doublet, t triplet, q quartet, quint quintet, sept septet, dd doublet of doublets, dt, doublet of triplet, bs broad singlet), number of protons and coupling constant in Hertz. For spectra obtained in DMSO-d₆, CD₃OD, the residual protons (2.50 and 3.31 ppm respectively) are used as the reference.

Analytical thin-layer chromatography (TLC) is performed on commercial silica plates (Merck 60-F 254, 0.25 mm thickness); compounds are visualized by UV light (254 nm). Flash chromatography is performed using silica gel (Merck Kieselgel 60, 230-400 mesh).

An Agilent 1100 series liquid chromatograph/mass selective detector (LC/MSD) is used to monitor the progress of reactions and check the purity of products using 254 nm, 220 nm wavelengths, and electrospray ionization (ESI) positive mode. Mass spectra are obtained in ESI positive mode.

(5-Bromo-thiazol-2-yl)-aryl-amines are synthesized in two steps from aryl thioureas unless specifically noted. A typical procedure is exemplified below. A solution of arylthiourea (1.67 mmol) and 1,2-dichloro-1-ethoxy-ethane (0.286 g, 2.00 mmol) in ethanol (3 ml) is heated at 75° C. for 12 hours in a sealed vial with stirring. It is then concentrated in vacuum to give the crude arylthiazol-2-yl-amine as oil or solid. Crude arylthiazol-2-yl-amines are used without further purification in the next step. Pure arylthiazol-2-yl amines could be obtained by recrystallization from ethanol. To a stirring solution of crude arylthiazol-2-yl-amine (0.42 mmol) in acetic acid is added bromine in acetic acid (0.42 mmol). The reaction is further stirred for 1 hour at room temperature after which the solvent is then removed in vacuum. The resulting residue is purified by HPLC (C₁₈ column, eluted with CH₃CN/H₂O with 0.035% TFA) to give (5-bromo-thiazol-2-yl)-aryl-amine as a solid.

### Example 1

### (5-Chloro-thiazol-2-yl)-(4-fluoro-phenyl)-amine

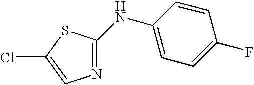

To a solution of (4-fluoro-phenyl)-thiazol-2-yl-amine (60 mg, 0.31 mmol) in THF (5 mL) is added N-chlorosuccinimide (42 mg, 0.31 mmol). The reaction is stirred at room temperature for 2 hours after which the solvent is removed in vacuum. The resulting residue is purified by HPLC (C₁₈ column, eluted with CH₃CN/H₂O with 0.035% TFA) to give the title compound as an off-white solid: ¹H NMR (DMSO-d₆) δ 7.15 (t, 2H, J=8.8 Hz), 7.23 (s, 1H), 7.58 (dd, 2H, J₁=4.8 Hz, J₂=8.8 Hz), 10.31 (s, 1H); m/z [M⁺+1] 228.9.

### Example 2

### (4-Fluoro-phenyl-(5-iodo-thiazol-2-yl)-amine

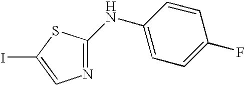

To a solution of (4-fluoro-phenyl)-thiazol-2-yl-amine (60 mg, 0.31 mmol) in THF (5 ml) is added N-iodo succinimide (70 mg, 0.31 mmol). The reaction is stirred at room temperature for 2 hours after which the solvent is removed in vacuum. The resulting residue is purified by HPLC (C₁₈ column, eluted with CH₃CN/H₂O with 0.035% TFA) to give the title compound as an off-white solid: ¹H NMR (DMSO-d₆) δ 7.14 (t, 2H, J=8.8 Hz), 7.32 (s, 1H), 7.58 (dd, 2H, J₁=4.8 Hz, J₂=8.8 Hz), 10.34 (s, 1H); m/z [M⁺1] 320.9.

### Example 3

### (5-Bromo-thiazol-2-yl)-(4-fluoro-phenyl)-methyl-amine

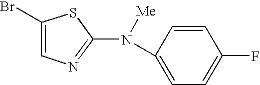

A solution of (4-fluoro-phenyl)-thiazol-2-yl-amine (97 mg, 0.5 mmol) in DMF (1 ml) is cooled to 0° C. and NaH (60% dispersion in mineral oil, 40 mg, 1.0 mmol) is added. The resulting mixture is stirred at 0° C. for 10 minutes prior to the addition of iodomethane (142 mg, 1.0 mmol). After stirring at room temperature for 12 hours, the reaction mixture is quenched with saturated aqueous NH₄Cl (10 ml) and extracted with ethyl acetate (10 ml×2). The organic layers are combined, washed with water (10 ml), saturated brine (10 ml) and dried with anhydrous Na₂SO₄. Following removal of the solvent in vacuum, the resulting (4-fluoro-phenyl)-methyl-thiazol-2-yl-amine is then subjected to the standard bromination procedure to give the title compound as an off-white solid after HPLC purification: ¹H NMR (DMSO-d₆) δ 3.39 (s, 3H), 7.26 (s, 1H), 7.31 (t, 2H, J=8.8 Hz), 7.52 (dd, 2H, J₁=5.2 Hz, J₂=8.8 Hz); m/z [M⁺+1] 286.9.

### Example 4

### N-(5-Bromo-thiazol-2-yl)-benzene-1,4-diamine

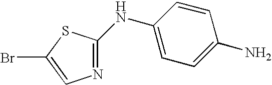

A solution of (4-nitro-phenyl)-thiazol-2-yl-amine (60 mg, 0.2 mmol) and SnCl₂.2H₂O (185 mg, 0.8 mmol) in ethanol (5 ml) is heated at 75° C. for 4 hours. The solvent is removed in vacuum and saturated aqueous Na₂CO₃ (5 mL) and ethyl acetate (5 mL) are added. The resulting mixture is filtered through celite and the ethyl acetate layer is separated, dried and concentrated in vacuum. The residue is purified by HPLC (C₁₈ column, eluted with CH₃CN/H₂O with 0.035% TFA) to give the title compound as a solid: ¹H NMR (DMSO-d₆) δ 7.18 (d, 2H, J=8.8 Hz), 7.31 (s, 1H), 7.59 (d, 2H, J=8.8 Hz), 9.20 (bs, 2H), 10.41 (s, 1H); m/z [M⁺+1] 269.9.

### Example 5

### N-[4-(5-Bromo-thiazol-2-ylamino)-phenyl]-benzamide

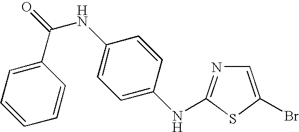

(4-Nitro-phenyl)-thiazol-2-yl-amine (200 mg) is dissolved in MeOH (30 ml) and hydrogenated at 50 psi in the presence of Pd/C (10%, 250 mg) for 12 hours. The catalyst is filtered off and the solvent is removed to afford N-thiazol-2-yl-benzene-1,4-diamine (0.16 g, 92%). N-thiazol-2-yl-benzene-1,4-diamine (30 mg, 0.16 mmol) is treated with benzoyl chloride (24 mg, 0.17 mmol) in the presence of triethylamine (32 mg, 0.31 mmol) in CH₂Cl₂ (2 ml) for 2 hours. The reaction is quenched with saturated aqueous Na₂CO₃ (10 ml) and extracted with ethyl acetate (10 ml×2). The ethyl acetate layers are combined and dried with anhydrous Na₂SO₄. N-[4-(Thiazol-2-ylamino)-phenyl]-benzamide is obtained after the removal of the solvent in vacuum. The crude thiazole is then subjected to the standard bromination procedure to afford the title compound as a white solid after HPLC purification: ¹H NMR (DMSO-d₆) δ 7.29 (s, 1H), 7.48-7.60 (m, 5H), 7.71 (d, 2H, J=7.2 Hz), 7.95 (d, 2H, J=7.2 Hz), 10.18 (s, 1H), 10.29 (s, 1H); m/z [M⁺1] 373.9.

### Example 6

### 4-(5-Bromo-thiazol-2-ylamino)-benzoic acid

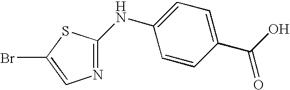

A solution of 4-(thiazol-2-ylamino)-benzoic acid ethyl ester (0.15 g, 0.60 mmol) and KOH (0.14 g, 2.4 mmol) in dioxane-H₂O (1:1, 20 ml) is stirred at room temperature for 24 hours. The reaction mixture is then acidified with hydrochloric acid until pH=4-5 (monitored with pH paper). The resulting precipitate is collected by filtration to give 4-(thiazol-2-ylamino)-benzoic acid as an off-white solid 4-(thiazol-2-ylamino)-benzoic acid is subjected to the standard bromination procedure to give the title compound: ¹H NMR (DMSO-d₆) δ 7.39 (s, 1H), 7.65 (d, 2H, J38.8 Hz), 7.88 (d, 2H, J=8.8 Hz), 10.71 (s, 1H), 12.56 (bs, 1H); m/z [M⁺1] 298.9.

### Example 7

### 4-(5-Bromo-thiazol-2-ylamino)-N-(2-morpholin-4-yl-ethyl)-benzamide TFA salt

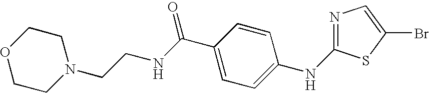

To a solution of 4-(5-bromo-thiazol-2-ylamino)-benzoic acid (24, 60 mg, 0.20 mmol), 2-morpholin-4-yl-ethylamine (78 mg, 0.6 mmol), and N,N-diisopropylethyl-amine (77 mg, 0.6 mmol) in DMF (2 ml) is added HATU (91 mg, 0.24 mmol). The resulting solution is stirred for 1 hour at room temperature and the solvent is removed in vacuum. The residue is purified by HPLC (C₁₈ column, eluted with CH₃CN/H₂O with 0.035% TFA) to give the title compound as a TFA salt: ¹H NMR (DMSO-d₆) δ 3.08-3.18 (m, 2H), 3.24-3.28 (m, 2H), 3.50-3.70 (m, 4H), 3.96-4.04 (m, 4H), 7.38 (s, 1H), 7.65 (d, 2H, J=8.8 Hz), 7.83 (d, 2H, J=8.8 Hz), 8.57 (t, 1H, J=6.0 Hz), 9.54 (bs, 1H), 10.64 (s, 1H); m/z [M⁺1] 411.0.

### Example 8

### N-(5-Bromo-thiazol-2-yl)-benzamide

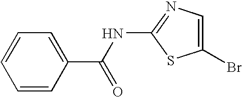

To a solution of 2-aminothiazole (0.10 g, 1.0 mmol), benzoic acid (0.15 g, 1.2 mmol), N,N-diisopropylethylamine (0.39 g, 3.0 mmol) in DMF (5 ml) is added HATU (0.46 g, 1.2 mmol). The reaction mixture is stirred at room temperature for 1 hour after which the solvent is removed in vacuum. Saturated aqueous NH₄Cl (10 ml) is added and the mixture is extracted with CH₂Cl₂ (10 ml). The CH₂Cl₂ layer is washed with saturated aqueous NaHCO₃ and dried with anhydrous Na₂SO₄. The solvent is removed in vacuum and resulting crude material is purified by column chromatography (silica gel, hexane-ethyl acetate) to give N-thiazol-2-yl-benzamide as a white solid. N-thiazol-2-yl-benzamide is subjected to the standard bromination procedure to give the title compound after HPLC purification: ¹H NMR (DMSO-d₆) δ 7.55 (t, 2H, J=7.2 Hz), 7.65 (t, 1H, J=7.2 Hz), 7.66 (s, 1H), 8.08 (d, 2H, J=6.4 Hz), 12.82 (bs, 1H); m/z [M⁺+1] 282.9.

### Example 9

### 1-(5-Bromo-thiazol-2-yl)-3-phenyl-urea

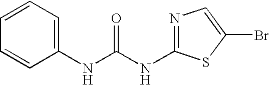

To a solution of 2-aminothiazole (0.1 g, 1.0 mmol) and N,N-diisopropylethylamine (0.39 g, 3.0 mmol) in THF (10 ml) is added phenyl isocyanate (0.12 g, 1.0 mmol). The reaction mixture is stirred at room temperature for 2 hours and then saturated aqueous NH₄Cl (10 ml) is added. The mixture is extracted with ethyl acetate (10 ml×2). The ethyl acetate layers are combined and dried with anhydrous Na₂SO₄. The solvent is evaporated in vacuum to afford 1-phenyl-3-thiazol-2-yl-urea as a white solid 1-phenyl-3-thiazol-2-yl-urea is subjected to the standard bromination procedure to give the title compound after HPLC purification. ¹H NMR (DMSO-d₆) δ 7.05 (t, 1H, J=8.0 Hz), 7.21 (t, 2H, J=8.0 Hz), 7.43-7.48 (m, 3H), 8.92 (s, 1H), 10.71 (s, 1H); m/z [M⁺+1] 297.9.

### Example 10

### (5-Bromo-thiazol-2-yl)-pyridin-2-yl-amine

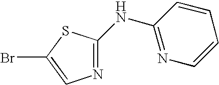

A solution of 2-chloropyridine (0.34 g, 3.0 mmol), 2-aminothiazole (0.314 g, 3.1 mmol), Na₂CO₃ (0.76 g, 7.2 mmol), Pd₂(dba)₃ (0.275 g, 0.3 mmol) and XantPhos (0.52 g, 0.9 mmol), H₂O (54 mg, 3.0 mmol) in toluene (25 ml) is heated at 100° C. for 12 hours. The reaction mixture is filtered and then concentrated in vacuum. The residue is purified by column chromatography (silica gel, eluted with CH₂Cl₂:MeOH:NH₃ (7N, in MeOH)=20:1:1) to give pyridin-2-yl-thiazol-2-yl-amine, which is then subjected to the standard bromination procedure to give the title compound as an off-white solid after HPLC purification: ¹H NMR (DMSO-d₆) δ 6.95 (t, 1H, J=6.4 Hz), 7.03 (d, 1H, J=8.0 Hz), 7.44 (s, 1H), 7.73 (t, 1H, J=6.4 Hz), 8.30 (d, 1H, J=3.6 Hz), 11.51 (s, 1H); m/z [M⁺+1] 255.9.

### Example 11

### (5-Bromo-thiazol-2-yl)-pyridin-3-yl-amine

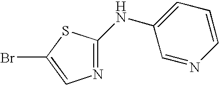

3-Pyridylthiourea (0.153 g, 1.0 mmol) and chloro-acetaldehyde solution in water (50%, 0.127 ml) are dissolved in ethanol (30 ml) and stirred for 16 hours at 60° C. The solvent is removed in vacuum and the resulting residue is subjected to the standard bromination procedure. The title compound is obtained after HPLC purification as an off-white solid: ¹H NMR (DMSO-d₆) δ 7.93 (s, 1H), 7.67 (dd, 1H, J₁=5.2 Hz, J₂=9.0 Hz), 8.28 (dd, 1H, J₁=2.4 Hz, J₂=9.0 Hz), 8.35 (d, 1H, J=5.2 Hz), 8.99 (d, 1H, J=2.0 Hz), 11.00 (s, 1H); m/z [M⁺+1] 255.9.

By repeating the procedures described in the examples, using appropriate starting materials, the following compounds of Formula I, as identified in Table 1, are obtained.

TABLE 1

| | | Physical Data |
|---|---|---|
| | | ¹H NMR 400 MHz |
| Compound | | (DMSO-d₆) and/or |
| Number | Structure | MS (m/z) |
| 12 | 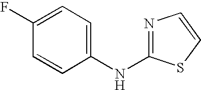 | ¹H NMR (DMSO-d₆) δ6.96 (d, 1 H, J = 4.0 Hz),7.21 (t, 2 H, J = 8.8 Hz),7.29 (d, 1 H, J = 8.0Hz),7.60 (dd, 2 H, J₁ = 4.0 Hz,J₂ = 8.0 Hz), 11.10(bs, 1 H); m/z [M⁺ + 1]195.0. |
| 13 | 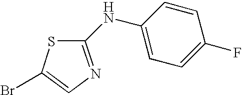 | ¹H NMR (DMSO-d₆) δ7.14 (t, 2 H, J = 8.8 Hz),7.28 (s, 1 H), 7.58 (dd,2 H, J₁ = 4.4 Hz, J₂ = 8.8 Hz),10.38 (s, 1 H); m/z[M⁺ + 1] 272.9. |
| 14 | 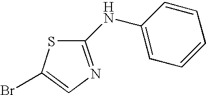 | ¹H NMR (DMSO-d₆) δ6.95 (t, 1 H, J = 7.2 Hz),7.28(s, 1 H), 7.29 (t, 2 H,7.6 Hz), 7.57 (d, 2 H,J = 7.6 Hz), 10.47 (bs,1 H); m/z [M⁺ + 1] 254.9. |
| 15 | 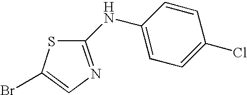 | ¹H NMR (DMSO-d₆) δ7.32 (s, 1 H), 7.34 (d,2 H, J = 7.8 Hz), 7.60 (d,2 H, J = 7.8Hz), 10.48(s, 1 H); m/z [M⁺ + 1]290.9. |
| 16 | 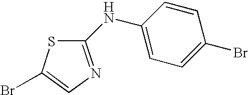 | ¹H NMR (DMSO-d₆) δ7.31 (s, 1 H), 7.46 (d,2 H, J = 8.8 Hz), 7.56 (d,2 H, J = 8.8 Hz), 10.54(s, 1 H); m/z [M⁺ + 1]334.8. |
| 17 | 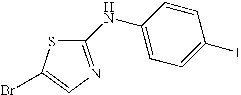 | ¹H NMR (DMSO-d₆) δ7.32 (s, 1 H), 7.41 (d,2 H, J = 8.8 Hz), 7.61 (d,2 H, J = 8.8 Hz), 10.44(s, 1 H); m/z [M⁺ + 1]380.8. |
| 18 | 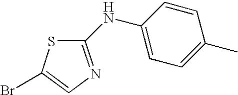 | ¹H NMR (DMSO-d₆) δ2.24(s, 3 H), 7.11 (d,2 H, J = 8.0 Hz), 7.27 (s,1 H), 7.44 (d, 2 H, J = 8.0 Hz),10.26 (s, 1 H); m/z[M⁺ + 1] 268.9. |
| 19 | 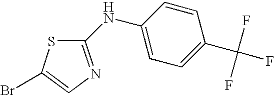 | ¹H NMR (DMSO-d₆) δ7.38 (s, 1 H), 7.64 (d,2 H, J = 8.0 Hz), 7.77 (d,2 H, J = 8.0 Hz), 11.83(bs, 1 H); m/z (M⁺ + 1]322.9. |
| 20 | 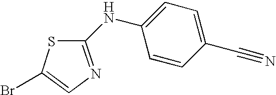 | ¹HNMR (DMSO-d₆) δ7.41 (s, 1 H), 7.74 (s,4 H), 10.94 (s, 1 H); m/z(M⁺ + 1] 279.9. |
| 21 | 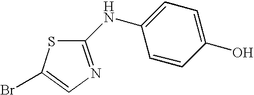 | ¹H NMR (DMSO-d₆) δ6.71 (d, 2 H, J = 8.8 Hz),7.20 (s, 1 H), 7.31 (d,2 H, J = 8.8 Hz), 9.96 (s,1 H); m/z [M⁺ + 1] 270.9. |
| 22 | 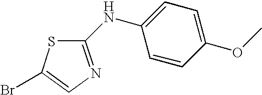 | ¹HNMR (DMSO-d₆) δ3.71 (s, 3 H), 6.89 (d,2 H, J = 8.8 Hz), 7.24 (s,1 H), 7.46 (d, 2 H, J = 8.8 Hz),10.19 (s, 1 H); m/z[M⁺ + 1] 284.9. |
| 23 | 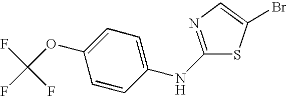 | ¹H NMR (DMSO-d₆) δ7.31 (d, 2 H, J = 8.8 Hz),7.33 (s, 1 H), 7.67 (d,2 H, J = 8.8 Hz), 10.57(s, 1 H); m/z [M⁺ + 1]338.9. |
| 24 | 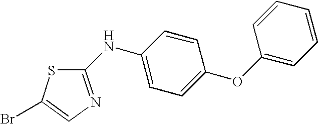 | ¹H NMR (DMSO-d₆) δ6.94 (d, 2 H, J = 8.8 Hz),7.01 (d, 2 H, J = 8.8 Hz),7.08 (t, 1 H, J = 7.2 Hz),7.27 (s, 1 H, 7.35 (t, 2 H,7.2 Hz), 7.59 (t, 2 H,J = 8.8 Hz), 10.41 (s,1 H; m/z [M⁺ + 1] 346.9. |
| 25 | 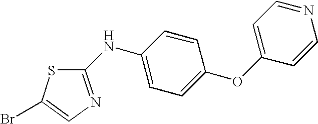 | ¹H NMR (DMSO-d₆) δ7.26 (d, 2 H, J = 9.0 Hz),7.33 (s, 1 H), 7.36 (d,2 H, J = 6.6 Hz), 7.74 (d,2 H, J = 9.0 Hz), 8.71 (d,2 H, J = 6.6 Hz), 10.58(s, 1 H); m/z [M⁺ + 1]347.9. |
| 26 | 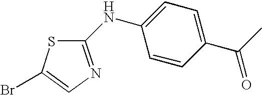 | ¹H NMR (DMSO-d₆) δ2.49 (s, 3 H), 7.40 (s,1 H), 7.37 (d, 2 H, J = 8.8 Hz),7.92 (d, 2 H, J = 8.8 Hz),10.75 (s, 1 H); m/z[M⁺ + 1] 296.9. |
| 27 | 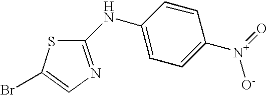 | ¹H NMR (DMSO-d₆) δ7.46 (s, 1 H), 7.79 (d,2 H, J = 9.6 Hz), 8.21 (d,2 H, J = 9.6 Hz), 11.19(s, 1 H); m/z [M⁺ + 1]299.9. |
| 28 | 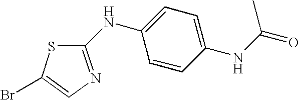 | ¹H NMR (DMSO-d₆) δ2.01 (s, 3 H), 7.26 (s,1 H), 7.45 (d, 2 H, J = 8.8 Hz),7.50 (d, 2 H, J = 8.8 Hz),9.83 (s, 1 H), 10.21(s, 1 H); m/z [M⁺ + 1]311.9. |
| 29 | 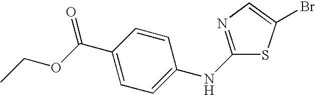 | ¹H NMR (DMSO-d₆) δ1.30 (t, 3 H, J = 7.6 Hz),4.27 (q, 2 H, J = 7.6 Hz),7.39 (s, 1 H), 7.69 (d,2 H, 8.8 Hz), 7.90 (d,2 H, J = 8.8 Hz), 10.80(s, 1 H); m/z [M⁺ + 1]326.9. |
| 30 | 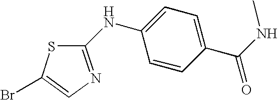 | ¹H NMR (DMSO-d₆) δ2.76 (d, 3 H, J = 4.4 Hz),7.36 (s, 1 H), 7.60 (d,2 H, J = 8.8 Hz), 7.79 (d,2 H, J = 8.8 Hz), 8.25 (q,1 H, J = 4.4 Hz), 10.57(s, 1 H); m/z [M⁺ + 1]311.9. |
| 31 | 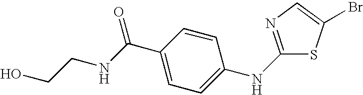 | ¹H NMR (DMSO-d₆) δ3.30 (q, 2 H, J = 6.0 Hz),3.49 (t, 2 H, J = 6.0 Hz),7.36 (s, 1 H), 7.60 (d,2 H, J = 7.8 Hz), 7.82 (d,2 H, J = 7.8 Hz), 8.27 (t,1 H, J = 6.0 Hz), 10.60(s, 1 H); m/z [M⁺ + 1]342.0. |
| 32 | 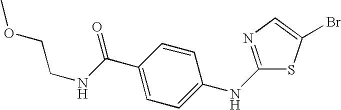 | ¹H NMR (DMSO-d₆) δ3.26 (s, 3 H), 3.36-3.48(m, 4 H), 7.36 (s, 1 H),7.61 (d, 2 H, J = 8.8 Hz),7.81 (d, 2 H, J = 8.8 Hz),8.34 (t, 1 H, J = 4.8 Hz),10.59 (s, 1 H); m/z[M⁺ + 1] 355.9. |
| 33 | 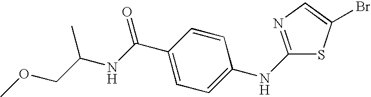 | ¹H NMR (DMSO-d₆) δ1.12 (d, 3 H), 3,24-3.28(dd, 1 H, J₁ = 6.6 Hz, J₂ =9.6 Hz), 3.26 (s, 3 H),3.39 (dd, 1 H, J₁ = 7.8 Hz,J₂ = 9.6 Hz), 4.18(sept, 1 H, J = 8.4 Hz),7.36 (s, 1 H), 7.61 (d,2 H, J = 7.8 Hz), 7.81 (d,2 H, J = 7.8 Hz), 8.03 (d,1 H, J = 6.6 Hz), 10.60(s, 1 H); m/z [M⁺ + 1]370.0. |
| 34 | 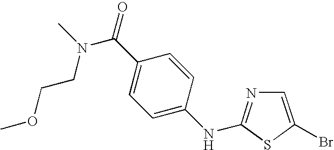 | ¹H NMR (DMSO-d₆) δ2.96 (s, 3 H), 3.16-3.32(m, 3 H), 3.40-3.60 (m,4 H), 7.34 (s, 1 H), 7.36(d, 2 H, J = 8.4 Hz), 7.60(d, 2 H, J = 8.4 Hz),10.54 (s, 1 H); m/z[M⁺ + 1] 370.0. |
| 35 | 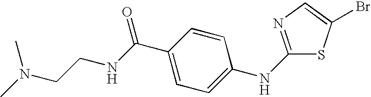 | ¹H NMR (DMSO-d₆) δ2.84 (d, 6 H, J = 4.2 Hz),3.24 (q, 2 H, J = 6.0 Hz),3.57 (q, 2 H, J = 6.0 Hz),7.38 (s, 1 H), 7.65 (d,2 H, J = 8.8 Hz), 7.83 (d,2 H, J = 8.8 Hz), 8.53 (t,1 H, J = 4.2 Hz), 9.23 (s,1 H), 10.65 (s, 1 H); m/z[M⁺ + 1] 369.0. |
| 36 | 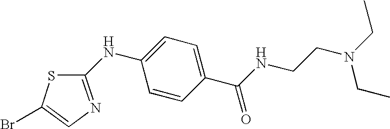 | ¹H NMR (DMSO-d₆) δ1.21 (t, 6 H, J = 7.2 Hz),3.18-3.26 (m, 6 H), 3.58(q, 2 H, J = 6.0 Hz), 7.38(s, 1 H), 7.65 (d, 2 H, J =9.0 Hz), 7.82 (d, 2 H, J =9.0 Hz), 8.56 (t, 1 H, J =4.8 Hz), 9.08 (s, 1 H),10.66 (s, 1 H); m/z[M⁺ + 1] 397.0. |
| 37 | 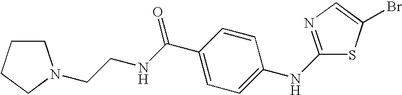 | ¹H NMR (DMSO-d₆) δ1.82-1.88 (m, 2 H), 1.99-2.05 (m, 2 H), 3.02-3.09(m, 2 H), 3.32 (q, 2 H, J =6.0 Hz), 3.57 (q, 2 H, J =6.0 Hz), 3.60-3.67 (m,2 H), 7.38 (s, 1 H), 7.65(d, 2 H, J = 8.4 Hz), 7.83(d, 2 H, J = 8.4 Hz), 8.55(t, 1 H, J = 6.0 Hz), 9.34(s, 1 H), 10.65 (s, 1 H);m/z [M⁺ + 1] 395.0. |
| 38 | 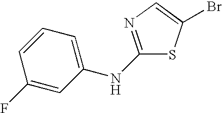 | ¹H NMR (DMSO-d₆) δ6.77 (td, 1 H, J = 2.8 Hz,J₂ = 8.4 Hz), 7.22(dd, 1 H, J₁ = 1.2 Hz, J₂ =8.4 Hz), 7.32 (q, 1 H, J =8.4 Hz), 7.35 (s, 1 H),6.63 (td, 1 H, J = 2.8 Hz,J₂ = 12 Hz), 10.60(s, 1 H); m/z [M⁺ + 1]272.9. |
| 39 | 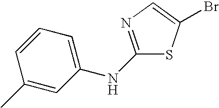 | ¹H NMR (DMSO-d₆) δ2.28 (s, 3 H), 6.78 (d,1 H, J = 8.0 Hz), 7.18 (t,1 H, J = 8.0 Hz), 7.29 (s,1 H), 7.32 (d, 1 H, J = 8.0 Hz,7.39 (s, 1 H), 10.24(s, 1 H); m/z [M⁺ + 1]268.9. |
| 40 | 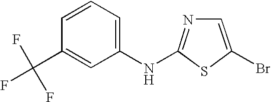 | ¹H NMR (DMSO-d₆) δ7.29 (d, 1 H, J = 8.0 Hz),7.39 (s, 1 H, 7.53 (t, 1 H,J = 8.0 Hz), 7.69 (d, 1 H,J = 8.0 Hz), 8.14 (s,1 H), 10.73 (s, 1 H); m/z[M⁺ + 1] 322.9. |
| 41 | 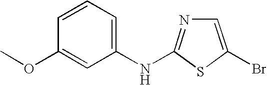 | ¹H NMR (DMSO-d₆) δ3.73 (s, 3 H), 6.55 (dd,1 H, J₁ = 2.4 Hz, J₂ = 8.0 Hz,7.05 (dd, 1 H, J₁ =2.4 Hz, J₂ = 8.0 Hz),7.20 (t, 1 H, J = 8.0 Hz),7.26 (t, 1 H, J = 2.0 Hz),7.30 (s, 1 H), 10.32 (s,1 H); m/z [M⁺ + 1] 384.9. |
| 42 | 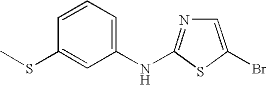 | ¹H NMR (DMSO-d₆) δ2.45 (s, 3 H), 6.85 (d,1 H, J = 7.2 Hz), 7.20-7.28 (m, 2 H), 7.32 (s,1 H), 7.56 (s, 1 H), 10.36(s, 1 H); m/z [M⁺ + 1]300.9. |
| 43 | 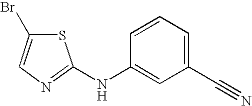 | ¹H NMR (DMSO-d₆) δ7.40 (s, 1 H), 7.42 (s,1 H), 7.51 (t, 1 H, J = 8.0 Hz),7.73 (dd, 1 H, J₁ =1.2 Hz, J₂ = 8.0 Hz),8.15 (s, 1 H), 10.71 (s,1 H); m/z [M⁺ + 1] 279.9. |
| 44 | 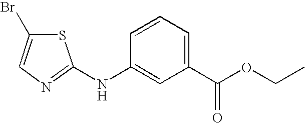 | ¹H NMR (DMSO-d₆) δ1.31 (t, 3 H, J = 7.2 Hz),4.31 (q, 2 H, J = 7.2 Hz),7.35 (s, 1 H), 7.44 (t, 1 H,J = 8.0 Hz), 7.54 (d, 1 H,J = 8.0 Hz),7.84 (dd,1 H, J₁ = 8.0 Hz, J₂= 1.6 Hz),8.20-8.24 (m, 1 H),10.60 (bs, 1 H); m/z[M⁺ + 1] 326.9. |
| 45 | 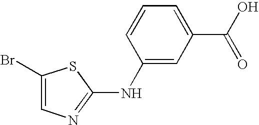 | ¹H NMR (DMSO-d₆) δ7.35 (s, 1 H), 7.42 (t, 1 H,J = 8.0 Hz), 7.53 (d, 1 H,J = 7.2 Hz), 7.76 (d, 1 H,J = 8.0 Hz), 8.25 (s,1 H), 10.57 (s, 1 H); m/z[M⁺ + 1] 298.9. |
| 46 | 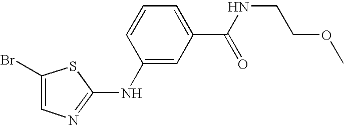 | ¹H NMR (DMSO-d₆) δ3.27 (s, 3 H), 3.38-3.48(m, 4 H), 7.33 (s, 1 H),7.35-7.42 (m, 2 H), 7.73(d, 1 H, J = 7.2 Hz), 7.98(s, 1 H), 8.44 (t, 1 H, J =5.2 Hz), 10.45 (s, 1 H);m/z [M⁺ + 1] 355.9. |
| 47 | 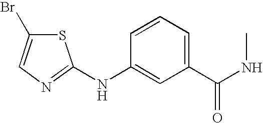 | ¹H NMR (DMSO-d₆) δ2.77 (d, 3 H, J = 3.6 Hz),7.33 (s, 1 H), 7.36-7.40(m, 2 H), 7,68-7.72 (m,1 H), 7.99 (s, 1 H), 8.36(d, 1 H, J = 3.6 Hz),10.44 (s, 1 H); m/z[M⁺ + 1] 311.9. |
| 48 | 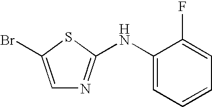 | ¹H NMR (DMSO-d₆) δ7.01 (dd, 1 H, J₁ = 7.2 Hz,J₂ = 14.8 Hz), 7.15(t, 1 H, J = 8.0 Hz), 7.24(dd, 1 H, J₁ = 8.0 Hz, J₂ =11.6 Hz), 7.31 (s, 1 H),8.31 (t, 1 H, J = 7.6 Hz),10.14 (s, 1 H); m/z[M⁺ + 1] 272.9. |
| 49 | 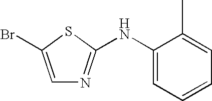 | ¹H NMR (DMSO-d₆) δ2.24 (s, 3 H), 7.01 (t, 1 H,J = 8.0 Hz), 7.17 (t, 1 H,J = 8.0 Hz), 7.20 (d, 1 H,J = 8.0 Hz), 7.21 (s,1 H), 7.78 (d, 1 H, J = 8.4 Hz),9.42 (s, 1 H); m/z[M⁺ + 1] 268.9. |
| 50 | 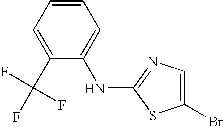 | ¹H NMR (DMSO-d₆) δ7.21 (s, 1 H), 7.33 (t, 1 H,J = 8.0 Hz), 7.65 (t, 1 H,J = 8.0 Hz), 7.71 (d, 1 H,J = 8.0 Hz), 7.86-7.88(m, 1 H), 9.61 (s, 1 H);m/z [M⁺ + 1] 322.9. |
| 51 | 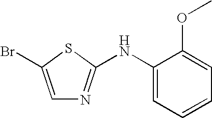 | ¹H NMR (DMSO-d₆) δ3.85 (s, 3 H), 6.90 (dt,1 H, J₁ = 8.0 Hz, J₂ = 1.6 Hz),6.97 (dt, 1 H, J₁ = 8.0 Hz, J₂ = 1.6 Hz),7.01 (dd, 1 H, J₁ = 8.0 Hz,J₂ = 1.6 Hz), 7.25(s, 1 H), 8.23 (dd, 1 H, J₁ =8.0 Hz, J₂ = 1.6 Hz),9.70 (s, 1 H); m/z[M⁺ + 1] 284.9. |
| 52 | 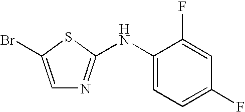 | ¹H NMR (DMSO-d₆) δ7.06 (t, 1 H, J = 8 Hz),7.28 (s, 1 H), 7.29-7.35(m, 1 H), 8.24-8.31 (m,1 H), 10.11 (s, 1 H); m/z[M⁺ + 1] 290.9. |
| 53 | 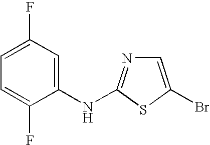 | ¹H NMR (DMSO-d₆) δ6.76-6.83 (m, 1 H), 7.25-7.32 (m, 1 H), 7.39 (s,1 H), 8.28-8.34 (m, 1 H),10.42 (s, 1 H); m/z[M⁺ + 1] 290.9. |
| 54 | 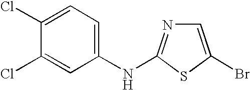 | ¹H NMR (DMSO-d₆) δ7.37 (s, 1 H), 7.42 (dd,1 H, J = 8.8 Hz, J₂ = 3.0 Hz),7.53 (d, 1 H, J = 8.8 Hz),8.06 (d, 1 H, J = 3.0 Hz),10.74 (bs, 1 H); m/z[M⁺ + 1] 324.8. |
| 55 | 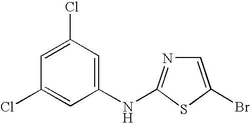 | ¹H NMR (DMSO-d₆) δ7.13 (t, 1 H, J = 1.2 Hz),7.40 (s, 1 H), 7.66 (d,2 H, J = 1.2 Hz), 10.86(s, 1 H); m/z [M⁺ + 1]324.8. |
| 56 | 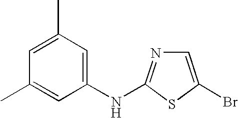 | ¹H NMR (DMSO-d₆) δ2.23 (s, 6 H), 6.61 (s,1 H), 7.16 (s, 2 H), 7.29(s, 1 H), 10.16 (s, 1 H);m/z [M⁺ + 1] 282.9. |
| 57 | 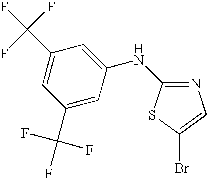 | ¹H NMR (DMSO-d₆) δ7.48 (s, 1 H), 7.61 (s,1 H), 8.24 (s, 2 H), 11.16(s, 1 H); m/z [M⁺ + 1]390.9. |
| 58 | 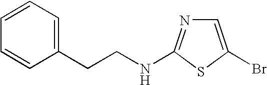 | ¹H NMR (DMSO-d₆) δ1.06 (t, 2 H, J = 7.6 Hz),2.84 (t, 2 H, J = 7.6 Hz),7.09 (s, 1 H), 7.18-7.33(m, 5 H), 8.01 (s, 1 H);m/z [M⁺ + 1] 282.9. |
| 59 | 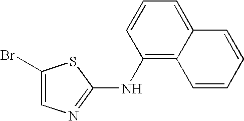 | ¹H NMR (DMSO-d₆) δ7.29 (s, 1 H), 7.49 (t, 1 H,J = 8.0 Hz), 7.52-7.60(m, 2 H), 7.67 (d, 1 H, J =8.8 Hz), 7.92-7.96 (m,1 H), 8.10 (d, 1 H, J = 7.2 Hz),8.21 (d, 1 H, J = 7.2 Hz),10.42 (s, 1 H); m/z[M⁺ + 1] 306.9. |
| 60 | 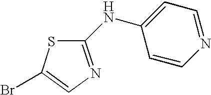 | ¹H NMR (DMSO-d₆) δ7.66 (s, 1 H), 7.94 (d,2 H, J = 5.6 Hz), 8.59 (d,2 H, J = 7.6 Hz), 12.16(bs, 1 H); m/z [M⁺ + 1]255.9. |

### Example 61

### Preparation of 4-bromo-aminothiazole

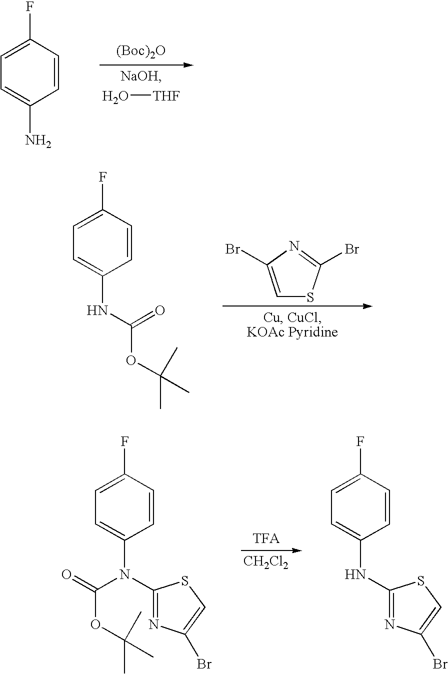

(4-Fluoro-phenyl-carbamic acid tert-butyl ester

To a solution of 4-fluoro-phenylamine (1.11 g, 10 mmol) in THF (30 ml) is added 1 N NaOH aqueous solution (30 ml). It is cooled to 0° C. using an ice bath and then (Boc)₂O (2.8 g, 12.8 mmol) is added in portions. The solution is stirred at room temperature for 16 hours and extracted with EtOAc (30 ml×2). The organic layers are combined, washed with 1N HCl, and then saturated NaHCO₃. The solvent is removed in vacuum and the residue is purified by column chromatography (silica gel, hexanes-EtOAc). The desired produce is obtained as an off-white solid: ¹H NMR (DMSO-d₆) δ 1.46 (s, 9H), 7.08 (t, 2H, J=8.8 Hz), 7.42-7.48 (m, 2H), 9.37 (sb, 1H); m/z [M⁺+2-t-Bu] 156.0.

### (4-Bromo-thiazol-2-yl)-(4-fluoro-phenyl)-carbamic acid tert-butyl ester

A mixture of (4-fluoro-phenyl)-carbamic acid tert-butyl ester (260 mg, 1.23 mmol), 2,4-dibromothiazole (100 mg, 0.41 mmol), copper powder (26 mg, 0.41 mmol), CuCl (41 mg, 0.41 mmol), and KOAc (40 mg, 0.41 mmol) in pyridine (4 ml) is heated at 100° C. for 2 hours. The cooled mixture is diluted with EtOAc (30 ml) and washed with H₂O (30 ml). The organic layer is dried over Na₂SO₄, filtered and concentrated in vacuum. The residue is purified by HPLC (C₁₈ column, eluted with CH₃CN/H₂O with 0.035% TFA) to give the title compound as solid: ¹H NMR (DMSO-d₆) δ 1.36 (s, 9H), 7.29 (t, 2H, J=8.8 Hz), 7.37-7.43 (m, 3H); m/z [M⁺2-t-Bu] 316.9.

### (4-Bromo-thiazol-2-yl)-(4-fluoro-phenyl-amine

To a solution of (4-bromo-thiazol-2-yl)-(4-fluoro-phenyl)-carbamic acid tert-butyl ester (10 mg, 0.027 mmol) in methylene chloride (5 ml) is added TFA (1 ml). The reaction is stirred at room temperature for 2 hours and concentrated. The residue is purified by HPLC (C₁₈ column, eluted with CH₃CN/H₂O with 0.035% TFA) to give the title compound as solid: ¹H NMR (DMSO-d₆) δ 6.96 (s, 1H), 7.18 (t, 2H, J=8.8 Hz), 7.55-7.59 (m, 2H), 10.50 (s, 1H); m/z [M⁺1] 273.0.

### Assays

Compounds of the present invention are assayed to measure their capacity to selectively inhibit Aurora kinases. In addition, compounds are assayed to measure their capacity to inhibit Abl, Bcr-Abl, Bmx, CDK1/cyclinB, CHK2, Fes, FGFR3, Flt3, GSK3β, JNK1α1, Lck, MKK4 and TrkB kinases.

### Aurora Kinase

Kinase activity is performed in 384 well ProxiPlates using 0.1 μg of kinase per well in kinase buffer (50 mM OPS pH 7.0, 10 mM MgCl₂, 1 mM DTT). Compounds are transferred to each well to a final concentration of 10 μM and kinase buffer containing 1 μM ATP, ³³Pγ-ATP. For IC₅₀ determination the final concentration of 10 μM ATP is used. Plates are incubated for 1 hour at room temperature before terminating the reaction with 1M ATP, 1 mM EDTA and 50 mg/ml SPA beads (Amersham/Pharmacia/GE health) and counted on TopCount. Compounds with more than 50% inhibition of kinase activity are retested to determine IC₅₀.

Cellular assay: Histone H3 ser10 phosphorylation: Fifty thousand HeLa cells plated in 12 well plates were treated with 100 ng/ml nocodazole for 20 hours prior to 1 hour incubation with compound. Cells are lysed in 2× sample buffer. Samples of total cell extracts, equal to one third of the cells per well, are subjected to SDS-PAGE and western blotting with anti phospho serine 10 histone H3 (Cell Signaling) to determine phosphorylation state.

FACS analysis: HeLa are treated with compound for various periods of time. Cells are trypsinized, washed once in PBS and fixed for 20 minutes at −20° C. Cells are resuspended in PBS, 1 mM EDTA, 100 μg/ml RNase and incubated for 30 minutes at 37° C., prior to addition of 10 μg/ml final concentration of propidium iodide (PI). Cell cycle distribution is determined on Beckman FACScalibur™ (BD Biosciences) and analyzed on FlowJo™ (Treestar).

High throughput microscopy (HTM): Alternatively cell cycle distribution is quantified using a confocal microscope system with capable of imaging a 384-well plate as follows. Four thousand HeLa cells are plated in each well of a 384 well plate, 24 hours later varying concentrations of compounds are added and plates are fixed in 4% paraformaldehyde and stained with DAPI. Automated acquisition of image from each well is performed and analyzed on EIDAQ 100 High Throughput Microscopy (HTM) (Q3DM/Beckman Coulter).

Cell proliferation assay: Cells are plated in 96-well plates and subjected to serial dilutions of compound. Forty eight hours later cell viability is measured using CellTiter96® (Promega) following the manufacture's protocol.

Microscopy: Hela cells are plated on cover slips, and subjected to various compound treatments 20 hours later. Compound treatments are terminated by washing the cover slips twice with PBS and fixing them in 4% paraformaldehyde at 37° C. for 10 minutes. The cover slips are washed in PBS and subsequently incubated in blocking solution (PBS 0.1% triton X-100 and 5% BSA) for a minimum of 1 hour. Cover slips are stained for 1 hour with 1:300 anti-phospho serine 10 histone H3 in blocking solution, 5% BSA, followed by incubation for 1 hour 1:1000 anti rabbit Cy3 in blocking solution. In some cases cover slips are also stained with FITC labeled anti-B-tubulin in blocking solution.

### Inhibition of Cellular BCR-Abl Dependent Proliferation (High Throughput Method)

The murine cell line used is the 32D hemopoietic progenitor cell line transformed with BCR-Abl cDNA (32D-p210). These cells are maintained in RPMI/10% fetal calf serum (RPMI/FCS) supplemented with penicillin 50 μg/mL, streptomycin 50 μg/mL and L-glutamine 200 mM. Untransformed 32D cells are similarly maintained with the addition of 15% of WEHI conditioned medium as a source of IL3.

50 μl of a 32D or 32D-p210 cells suspension are plated in Greiner 384 well microplates (black) at a density of 5000 cells per well. 50 nl of test compound (1 mM in DMSO stock solution) is added to each well (STI571 is included as a positive control). The cells are incubated for 72 hours at 37° C., 5% CO₂. 10 μL of a 60% Alamar Blue solution (Tek diagnostics) is added to each well and the cells are incubated for an additional 24 hours. The fluorescence intensity (Excitation at 530 nm, Emission at 580 nm) is quantified using the Acquest™ system (Molecular Devices).

### Inhibition of Cellular BCR-Abl Dependent Proliferation

32D-p210 cells are plated into 96 well TC plates at a density of 15,000 cells per well. 50 μL of two fold serial dilutions of the test compound (Cₘₐₓ is 40 μM) are added to each well (STI571 is included as a positive control). After incubating the cells for 48 hours at 37° C., 5% CO₂, 15 μL of MTT (Promega) is added to each well and the cells are incubated for an additional 5 hours. The optical density at 570 nm is quantified spectrophotometrically and IC₅₀ values, the concentration of compound required for 50% inhibition, determined from a dose response curve.

### Effect on Cell Cycle Distribution

32D and 32D-p210 cells are plated into 6 well TC plates at 2.5×10⁶ cells per well in 5 ml of medium and test compound at 1 or 10 μM is added (STI571 is included as a control). The cells are then incubated for 24 or 48 hours at 37° C., 5% CO₂. 2 ml of cell suspension is washed with PBS, fixed in 70% EtOH for 1 hour and treated with PBS/EDTA/RNase A for 30 minutes. Propidium iodide (Cf-10 μg/ml) is added and the fluorescence intensity is quantified by flow cytometry on the FACScalibur™ system (BD Biosciences). Test compounds of the present invention demonstrate an apoptotic effect on the 32D-p210 cells but do not induce apoptosis in the 32D parental cells.

### Effect on Cellular BCR-Abl Autophosphorylation

BCR-Abl autophosphorylation is quantified with capture Elisa using a c-abl specific capture antibody and an antiphosphotyrosine antibody. 32D-p210 cells are plated in 96 well TC plates at 2×10⁵ cells per well in 50 μL of medium. 50 μL of two fold serial dilutions of test compounds (Cₘₐₓ is 10 μM) are added to each well (STI571 is included as a positive control). The cells are incubated for 90 minutes at 37° C., 5% CO₂. The cells are then treated for 1 hour on ice with 150 μL of lysis buffer (50 mM Tris-HCl, pH 7.4, 150 mM NaCl, 5 mM EDTA, 1 mM EGTA and 1% NP-40) containing protease and phosphatase inhibitors. 50 μL of cell lysate is added to 96 well optiplates previously coated with anti-abl specific antibody and blocked. The plates are incubated for 4 hours at 4° C. After washing with TBS-Tween 20 buffer, 50 μL of alkaline-phosphatase conjugated anti-phosphotyrosine antibody is added and the plate is further incubated overnight at 4° C. After washing with TBS-Tween 20 buffer, 90 μL of a luminescent substrate are added and the luminescence is quantified using the Acquest™ system (Molecular Devices). Test compounds of the invention that inhibit the proliferation of the BCR-Abl expressing cells, inhibit the cellular BCR-Abl autophosphorylation in a dose-dependent manner.

### Effect on Proliferation of Cells Expressing Mutant Forms of Bcr-abl

Compounds of the invention are tested for their antiproliferative effect on Ba/F3 cells expressing either wild type or the mutant forms of BCR-Abl (G250E, E255V, T315I, F317L, M351T) that confers resistance or diminished sensitivity to STI571. The antiproliferative effect of these compounds on the mutant-BCR-Abl expressing cells and on the non transformed cells were tested at 10, 3.3, 1.1 and 0.37 μM as described above (in media lacking IL3). The IC₅₀ values of the compounds lacking toxicity on the untransformed cells were determined from the dose response curves obtained as describe above.

### FGFR3 (Enzymatic Assay)

Kinase activity assay with purified FGFR3 (Upstate) is carried out in a final volume of 10 μL containing 0.25 μg/mL of enzyme in kinase buffer (30 mM Tris-HCl pH7.5, 15 mM MgCl₂, 4.5 mM MnCl₂, 15 μM Na₃VO₄ and 50 μg/mL BSA), and substrates (5 μg/mL biotin-poly-EY(Glu, Tyr) (CIS-US, Inc.) and 3 μM ATP). Two solutions are made: the first solution of 5 μl contains the FGFR3 enzyme in kinase buffer was first dispensed into 384-format ProxiPlate® (Perkin-Elmer) followed by adding 50 nL of compounds dissolved in DMSO, then 5 μl of second solution contains the substrate (poly-EY) and ATP in kinase buffer was added to each wells. The reactions are incubated at room temperature for one hour, stopped by adding 10 μL of HTRF detection mixture, which contains 30 mM Tris-HCl pH7.5, 0.5 M KF, 50 mM ETDA, 0.2 mg/mL BSA, 15 μg/mL streptavidin-XL665 (CIS-US, Inc.) and 150 ng/mL cryptate conjugated anti-phosphotyrosine antibody (CIS-US, Inc.). After one hour of room temperature incubation to allow for streptavidin-biotin interaction, time resolved florescent signals are read on Analyst GT (Molecular Devices Corp.). IC₅₀ values are calculated by linear regression analysis of the percentage inhibition of each compound at 12 concentrations (1:3 dilution from 50 μM to 0.28 nM). In this assay, compounds of the invention have an IC₅₀ in the range of 10 nM to 2 μM.

### FGFR3 (Cellular Assay)

Compounds of the invention are tested for their ability to inhibit transformed Ba/F3-TEL-FGFR3 cells proliferation, which is depended on FGFR3 cellular kinase activity. Ba/F3-TEL-FGFR3 are cultured up to 800,000 cells/mL in suspension, with RPMI 1640 supplemented with 10% fetal bovine serum as the culture medium. Cells are dispensed into 384-well format plate at 5000 cell/well in 50 μL culture medium. Compounds of the invention are dissolved and diluted in dimethylsulfoxide (DMSO). Twelve points 1:3 serial dilutions are made into DMSO to create concentrations gradient ranging typically from 10 mM to 0.05 μM. Cells are added with 50 nL of diluted compounds and incubated for 48 hours in cell culture incubator. AlamarBlue® (TREK Diagnostic Systems), which can be used to monitor the reducing environment created by proliferating cells, are added to cells at final concentration of 10%. After additional four hours of incubation in a 37° C. cell culture incubator, fluorescence signals from reduced AlamarBlue® (Excitation at 530 nm, Emission at 580 nm) are quantified on Analyst GT (Molecular Devices Corp.). IC₅₀ values are calculated by linear regression analysis of the percentage inhibition of each compound at 12 concentrations.

### FLT3 and PDGFRβ (Cellular Assay)

The effects of compounds of the invention on the cellular activity of FLT3 are conducted using identical methods as described above for FGFR3 cellular activity, except that instead of using Ba/F3-TEL-FGFR3, Ba/F3-FLT3-ITD is used.

### Upstate KinaseProfiler™—Radio-Enzymatic Filter Binding Assay

Compounds of the invention are assessed for their ability to inhibit individual members of the kinase panel. The compounds are tested in duplicates at a final concentration of 10 μM following this generic protocol. Note that the kinase buffer composition and the substrates vary for the different kinases included in the “Upstate KinaseProfiler™” panel. Kinase buffer (2.5 μL, 10×—containing MnCl₂ when required), active kinase (0.001-0.01 Units; 2.5 μL), specific or Poly(Glu-4-Tyr) peptide (5-500 μM or 0.01 mg/ml) in kinase buffer and kinase buffer (50 μM; 5 μL) are mixed in an eppendorf on ice. A Mg/ATP mix (10 μL; 67.5 (or 33.75) mM MgCl₂, 450 (or 225) μM ATP and 1 μCi/μl [γ-³²P]-ATP (3000 Ci/mmol)) is added and the reaction is incubated at about 30° C. for about 10 minutes. The reaction mixture is spotted (20 μL) onto a 2 cm×2 cm P81 (phosphocellulose, for positively charged peptide substrates) or Whatman No. 1 (for Poly (Glu4-Tyr) peptide substrate) paper square. The assay squares are washed 4 times, for 5 minutes each, with 0.75% phosphoric acid and washed once with acetone for 5 minutes. The assay squares are transferred to a scintillation vial, 5 ml scintillation cocktail are added and ³²P incorporation (cpm) to the peptide substrate is quantified with a Beckman scintillation counter. Percentage inhibition is calculated for each reaction.

Compounds of Formula I, in free form or in pharmaceutically acceptable salt form, exhibit valuable pharmacological properties, for example, as indicated by the in vitro tests described in this application. For example, compounds of Formula I preferably show an IC₅₀ in the range of 1×10⁻¹⁰ to 1×10⁻⁵ M, preferably less than 1 μM, more preferably less than 250 nM, more preferably less than 1100 nM. Compounds of Formula I preferably, at a concentration of 10 μM, preferably show a percentage inhibition of greater than 50%, preferably greater than about 80%, against Abl, Aurora-A, Bcr-Abl, Bmx, CDK1/cyclinB, CHK2, Fes, FGFR3, Flt3, GSK3β, JNK1α1, Lck, MKK4 and TrkB kinases.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes.

We claim:

1. A compound of Formula I: 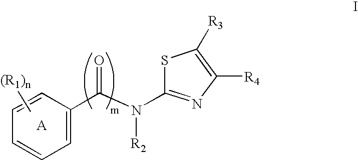
in which:
n is selected from 0, 1, 2 and 3;
m is selected from 0 and 1;
R₁ is selected from halo, cyano, hydroxy, nitro, C₁₋₆alkyl, C₁₋₆alkoxy, halo-substituted-C₁₋₆alkyl, halo-substituted-C₁₋₆alkoxy, —S(O)₀₋₂R₅, —NR₅R₅, —C(O)NR₅R₆, —C(O)NR₅R₆, —C(O)NR₅XOR₅, —C(O)NR₅XNR₅R₅, —OR₆, —C(O)OR₅, —NR₅C(O)R₆; wherein each R₅ is independently selected from hydrogen and C₁₋₆alkyl; and R₆ is selected from C₆₋₁₀aryl-C₀₋₄alkyl, C₁₋₁₀heteroaryl-C₀₋₄alkyl, C₃₋₁₂cycloalkyl-C₀₋₄alkyl and C₃₋₈heterocycloalkyl-C₀₋₄alkyl; or where n is 2, two adjacent R₁ radicals together with the atoms to which they are both attached, form phenyl;
R₂ is hydrogen and methyl;
R₃ is halo;
R₄ is selected from hydrogen, halogen and C₁₋₆alkyl; or R₃ and R₄ together with the atoms to which R₃ and R₄ are attached form phenyl; and ring A can optionally have up to three ═C— groups replaced with ═N—; and the pharmaceutically acceptable salts, hydrates, solvates and isomers thereof.

2. The compound of claim 1 in which ring A is selected from phenyl, pyridinyl and naphthyl; m is zero; R₃ is halo and R₄ is hydrogen.

3. The compound of claim 2 in which R₁ is selected from methyl, hydroxy, methoxy, chloro, fluoro, bromo, carboxy, amino, cyano, nitro, methyl-sulfanyl, trifluoromethoxy, trifluoromethyl, methyl-carbonyl, ethoxy-carbonyl, —C(O)NHR₆, —C(O)NH(CH₂)₂OCH₃, —C(O)NHCH(CH₃)CH₂OCH₃, —C(O)N(CH₃)(CH₂)₂OCH₃, —C(O)NH(CH₂)₂OH, —C(O)NH(CH₂)₂N(CH₃)₂, —C(O)NH(CH₂)₂N(C₂Hs)₂, —C(O)NHCH₃, —NHC(O)R₆, —NHC(O)CH₃ and —OR₆; wherein R₆ is selected from phenyl, morpholino-ethyl, pyridinyl and pyrrolidinyl-ethyl.

4. The compound of claim 1 selected from (5-Bromo-thiazol-2-yl)-p-tolyl-amine; 4-(5-Bromo-thiazol-2-ylamino)-phenol; (5-Bromo-thiazol-2-yl)-(4-methoxy-phenyl)-amine; 4-(5-Bromo-thiazol-2-ylamino)-benzoic acid; 4-(5-Bromo-thiazol-2-ylamino)-N-(2-morpholin-4-yl-ethyl)-benzamide; 4-(5-Bromo-thiazol-2-ylamino)-N-(2-methoxy-ethyl)-benzamide; (5-Bromo-thiazol-2-yl)-[4-(1-methylamino-vinyl)-phenyl]-amine; 3-(5-Bromo-thiazol-2-ylamino)-benzoic acid; N-[4-(5-Bromo-thiazol-2-ylamino)-phenyl]-benzamide; N-[4-(5-Bromo-thiazol-2-ylamino)-phenyl]-acetamide; 3-(5-Bromo-thiazol-2-ylamino)-N-(2-methoxy-ethyl)-benzamide; 3-(5-Bromo-thiazol-2-ylamino)-N-methyl-benzamide; (5-Bromo-thiazol-2-yl)-[4-(pyridin-4-yloxy)-phenyl]-amine; (5-Bromo-thiazol-2-yl)-(4-chlorophenyl)-amine; Benzothiazol-2-yl-(4-fluoro-phenyl)-amine; 4-(5-Bromo-thiazol-2-ylamino)-N-(2-hydroxy-ethyl)-benzamide; 4-(5-Bromo-thiazol-2-ylamino)-N-(2-dimethylamino-ethyl)-benzamide; 4-(5-Bromo-thiazol-2-ylamino)-N-(2-diethylamino-ethyl)-benzamide; N-(5-Bromo-thiazol-2-yl)-benzamide; (5-Bromo-thiazol-2-yl)-(3-fluoro-phenyl)-amine; (5-Bromo-thiazol-2-yl)-(3-trifluoromethyl-phenyl)-amine; (5-Bromo-thiazol-2-yl)-(3-methoxy-phenyl)-amine; (5-Bromo-thiazol-2-yl)-m-tolyl-amine; (5-Bromo-thiazol-2-yl)-pyridin-2-yl-amine; N-(5-Bromo-thiazol-2-yl)-benzene-1,4-diamine; 1-[4-(5-Bromo-thiazol-2-ylamino)-phenyl]-ethanone; 4-(5-Bromo-thiazol-2-ylamino)-benzoic acid ethyl ester; (5-Bromo-thiazol-2-yl)-pyridin-4-yl-amine; (5-Bromo-thiazol-2-yl)-pyridin-3-yl-amine; N-(5-Bromo-thiazol-2-yl)-benzamide; (5-Bromo-thiazol-2-yl)-(4-trifluoromethyl-phenyl)-amine; 3-(5-Bromo-thiazol-2-ylamino)-benzoic acid ethyl ester; (5-Bromo-thiazol-2-yl)-phenyl-amine; (5-Bromo-thiazol-2-yl)-(2-methoxy-phenyl)-amine; (5-Bromo-thiazol-2-yl)-(4-fluoro-phenyl)-amine; (5-Chloro-thiazol-2-yl)-(4-fluoro-phenyl)-amine; (4-Fluoro-phenyl)-(5-iodo-thiazol-2-yl)-amine; 4-(5-Bromo-thiazol-2-ylamino)-benzonitrile; (5-Bromo-thiazol-2-yl)-o-tolyl-amine; (5-Bromo-thiazol-2-yl)-naphthalen-1-yl-amine; (5-Bromo-thiazol-2-yl)-(2-fluoro-phenyl)-amine; 3-(5-Bromo-thiazol-2-ylamino)-benzonitrile; (5-Bromo-thiazol-2-yl)-(3-methylsulfanyl-phenyl)-amine; (4-Bromo-phenyl)-(5-bromo-thiazol-2-yl)-amine; (5-Bromo-thiazol-2-yl)-(4-phenoxy-phenyl)-amine; (5-Bromo-thiazol-2-yl)-(4-nitro-phenyl)-amine; 4-(5-Bromo-thiazol-2-ylamino)-N-(2-pyrrolidin-1-yl-ethyl)-benzamide; 4-(5-Bromo-thiazol-2-ylamino)-N-(2-methoxy-1-methyl-ethyl)-benzamide; and 4-(5-Bromo-thiazol-2-ylamino)-N-(2-methoxy-ethyl)-N-methyl-benzamide.

5. A pharmaceutical composition comprising a therapeutically effective amount of a compound of claim 1 in combination with a pharmaceutically acceptable excipient.

6. A method for treating a disease in an animal in which inhibition of kinase activity can prevent, inhibit or ameliorate the pathology and/or symptomology of the disease, which method comprises administering to the animal a therapeutically effective amount of a compound of claim 1.

7. The method of claim 6 in which the kinase is selected from Abl, Aurora-A, Bcr-Abl, Bmx, CDK1/cyclinB, CHK2, Fes, FGFR3, Flt3, GSK3β, JNK1α1, Lck, MKK4 and TrkB.

8. The use of a compound of claim 1 in the manufacture of a medicament for treating a disease in an animal in which the kinase activity of Abl, Aurora-A, Bcr-Abl, Bmx, CDK1/cyclinB, CHK2, Fes, FGFR3, Flt3, GSK3β, JNK1α1, Lck, MKK4 and TrkB contributes to the pathology and/or symptomology of the disease.
